(19) United States
(12) Patent Application Publication
(10) Pub. No.: US 2026/0102167 A1
(43) Pub. Date: Apr. 16, 2026

(54) CLIP UNIT

(71) Applicants: Haruhiro INOUE, Kanagawa (JP); OLYMPUS MEDICAL SYSTEMS CORP., Tokyo (JP)

(72) Inventors: Jun SHIRAI, Tachikawa-shi (JP); Masaru Yuasa, Hachioji-shi (JP); Haruhiro INOUE, Yokohama-shi (JP)

(21) Appl. No.: 19/420,054

(22) Filed: Dec. 15, 2025

Related U.S. Application Data
(63) Continuation of application No. PCT/JP2024/003143, filed on Jan. 31, 2024.
(60) Provisional application No. 63/508,592, filed on Jun. 16, 2023.

(51) Int. Cl. A61B 17/122

(52) U.S. Cl. CPC A61B 17/122

(57) ABSTRACT

A clip unit includes: a clip having a first arm and a second arm that are capable of opening and closing toward a distal end side; and a tube-shaped member capable of storing at least a part of the clip, in which the first arm has a first claw that extends to a closing side in an opening/closing direction at a distal end, and the first claw has a first anchor.

### CLAIM FOR PRIORITY

This application is a continuation of and claims the benefit of priority of International Application No. PCT/JP2024/003143, filed on Jan. 31, 2024, and published as WO2024/257386 on Dec. 19, 2024, which claims priority to U.S. Patent Application No. 63/508,592, filed Jun. 16, 2023; the benefit of priority of each of which is hereby claimed herein, and which applications and publication are hereby incorporated herein by reference in their entireties.

### BACKGROUND OF THE INVENTION

### Field of the Invention

The present invention relates to a clip unit.

### Description of Related Art

In endoscopic treatment, a clip unit capable of achieving hemostasis and the like by ligating a resected area after treatment and the like is used. The clip unit includes a clip that clamps a resected area and the like, a pressing tube that houses the clip and locks it in a closed state, and the like. The clip unit is introduced to a treatment position using a clip introduction device that can be inserted into and go through the channel of an endoscope.

Japanese Unexamined Patent Application, First Publication No. H9-38093 describes a treatment tool for puncturing biological tissue. The treatment tool described in Japanese Unexamined Patent Application, First Publication No. H9-38093 can be placed inside the body in a state in which it has punctured a biological tissue.

### SUMMARY OF THE INVENTION

In order to solve the problem described above, the present invention proposes the following means.

A clip unit according to a first aspect of the present invention includes: a clip having a first arm and a second arm that are capable of opening and closing toward a distal end side; and a tube-shaped member capable of storing at least a part of the clip, in which the first arm has a first claw that extends to a closing side in an opening/closing direction at a distal end, and the first claw has a first anchor.

### BRIEF DESCRIPTION OF THE DRAWINGS

FIG. 1 is a perspective view of a clip introduction device of a clip delivery device according to a first embodiment.

FIG. 2 is a perspective view of a clip unit of the clip delivery device.

FIG. 3 is a perspective view of the clip unit in which a pressing member is transparently shown.

FIG. 4 is a diagram of one pair of arms of the clip unit in a closed state that is seen in a longitudinal direction.

FIG. 5 is a diagram illustrating a modified example of a first anchor.

FIG. 6 is a diagram illustrating a modified example of the first anchor.

FIG. 7 is a diagram of the pair of arms in the closed state that is seen in an opposing direction.

FIG. 8 is a diagram of the pair of arms in the closed state that is seen in an opening/closing direction.

FIG. 9 is a cross-sectional view of the pressing member in the longitudinal direction.

FIG. 10 is a perspective view of a connecting member of the clip unit.

FIG. 11 is a diagram illustrating the clip unit loaded into the clip introduction device.

FIG. 12 is a diagram illustrating the clip unit introduced into the inside of the body.

FIG. 13 is a diagram illustrating the clip unit that grasps a first mucosa.

FIG. 14 is a diagram illustrating the first mucosa grasped by one pair of arms.

FIG. 15 is a diagram illustrating the clip unit in which a second claw has been pulled out from the first mucosa.

FIG. 16 is a diagram illustrating the first mucosa from which the second claw has been pulled out.

FIG. 17 is a diagram illustrating the clip unit that grasps a second mucosa.

FIG. 18 is a diagram illustrating the clip unit that has closed the pair of arms.

FIG. 19 is a diagram illustrating the clip unit with a clip locked.

FIG. 20 is a diagram illustrating the clip unit with the clip separated.

FIG. 21 is a diagram illustrating the clip unit after breakage.

FIG. 22 is a diagram illustrating a modified example of the second claw.

FIG. 23 is a diagram illustrating modified examples of the first claw and the second claw.

FIG. 24 is a diagram illustrating a modified example of the pressing member and a modified example of the pair of arms.

FIG. 25 is a diagram illustrating a modified example of the pressing member and a modified example of the pair of arms.

FIG. 26 is a diagram illustrating a modified example of the pressing member and a modified example of the pair of arms.

FIG. 27 is a diagram illustrating a modified example of the pressing member and a modified example of the pair of arms.

FIG. 28 is a diagram illustrating a clip unit according to a second embodiment.

FIG. 29 is a diagram illustrating the clip unit.

FIG. 30 is a diagram illustrating the clip unit.

FIG. 31 is a diagram illustrating a modified example of a clip of the clip unit.

FIG. 32 is a diagram illustrating a modified example of the clip.

FIG. 33 is a diagram illustrating a modified example of the clip.

FIG. 34 is a diagram illustrating the modified example.

FIG. 35 is a diagram illustrating a clip unit according to a third embodiment.

FIG. 36 is a diagram illustrating the clip unit.

FIG. 37 is a diagram illustrating the clip unit.

FIG. 38 is a diagram illustrating a modified example of the clip of the clip unit.

FIG. 39 is a diagram illustrating a clip unit according to a fourth embodiment.

FIG. 40 is a diagram illustrating the clip unit.

FIG. 41 is a diagram illustrating the clip unit.

FIG. 42 is a diagram illustrating a modified example of an inner face anchor.

FIG. 43 is a diagram illustrating a modified example of the clip.

FIG. 44 is a diagram illustrating modified examples of the first claw and the second claw.

FIG. 45 is a diagram illustrating modified examples of the first claw and the second claw.

FIG. 46 is a diagram illustrating modified examples of the first claw and the second claw.

### DETAILED DESCRIPTION OF THE INVENTION

### First Embodiment

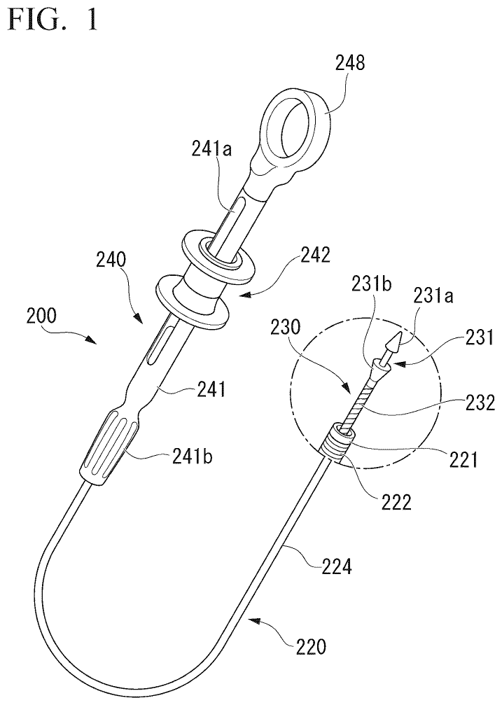

A clip delivery device 300 according to a first embodiment of the present invention is described with reference to FIGS. 1 to 21.

### Clip Delivery Device 300

The clip delivery device (a clip system) 300 includes a clip introduction device (applicator) 200 and a clip unit 100. The clip unit 100 is loaded into the clip introduction device 200.

### Clip Introduction Device 200

FIG. 1 is a perspective view of the clip introduction device 200.

The clip introduction device (applicator) 200 includes a sheath 220, an operation wire 230, and an operation unit 240. The clip introduction device 200 is, for example, inserted into and passes through a treatment tool insertion channel of an endoscope and used in combination with the endoscope. For this reason, the sheath 220 is formed to be sufficiently longer than the length of the treatment tool insertion channel of the endoscope. The sheath 220 has flexibility and bends in accordance with the curvature of an insertion portion of the endoscope.

The sheath 220 includes a distal-end tip 221, a distal-side coil 222, and a proximal-side coil 224 and is formed in an overall elongated tubular shape. The distal-side coil 222 is arranged on the distal end portion side of the sheath 220. The distal-end tip 221 is arranged at the distal end portion of the distal-side coil 222.

As shown in FIG. 1, the operation wire (power transmission section) 230 includes an arrowhead hook portion (connection section) 231, which is connected to the clip unit 100, and a wire 232 that operates the arrowhead hook portion 231.

The arrowhead hook portion 231 includes an approximately conical engaging portion 231a that engages with the clip unit 100 and a wire connection portion 231b disposed at the proximal end of the engaging portion 231a. The arrowhead hook portion 231 is formed, for example, of a metallic material such as stainless steel.

The wire 232 is inserted into and passes through the sheath 220 so as to be able to advance and retract. The distal end portion of the wire 232 is fixed to the proximal end of the wire connection portion 231b, for example, through welding.

As illustrated in FIG. 1, the operation unit 240 includes an operation unit main body 241, a slider 242, and a thumb ring 248. The operation unit main body 241 is formed, for example, through injection molding of a resin material. The operation unit main body 241 includes a slit portion 241a and a rotary grip 241b on its distal end side. The slit portion 241a supports the slider 242 such that it can advance and retract.

The slider 242 is attached to the operation unit main body 241 such that it can advance and retract in the direction of the longitudinal axis, and the proximal end of the wire 232 is attached thereto. As the slider 242 advances and retracts along the operation unit main body 241, the wire 232 advances and retracts relative to the sheath 220, thereby causing the arrowhead hook portion 231 to advance and retract.

The thumb ring 248 is attached to the proximal end of the operation unit main body 241 such that it is rotatable around the longitudinal axis of the operation unit main body 241.

### Clip Unit 100

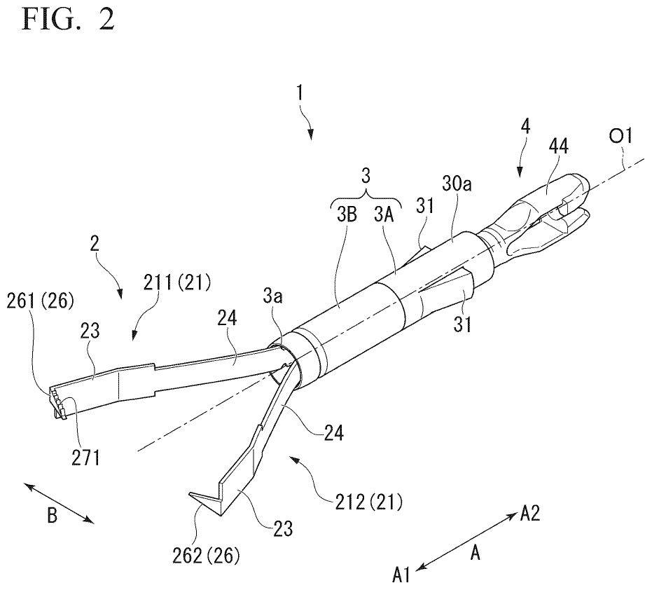

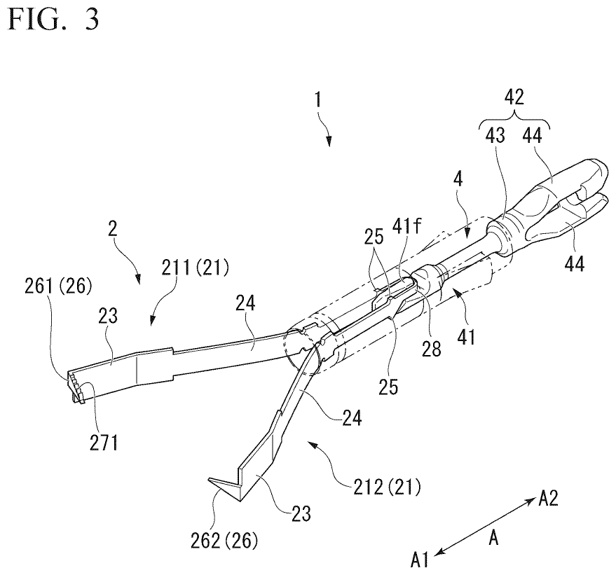

FIG. 2 is a perspective view of the clip unit 100. FIG. 3 is a perspective view of the clip unit 100 in which a pressing member 3 is transparently shown. The clip unit 100 includes a clip 2, the pressing member 3, and a connecting member 4.

In the following description, the side of the clip 2 in the longitudinal direction A of the clip unit 100 is referred to as a distal end side (remote side) A1 of the clip unit 100, and the side of the connecting member 4 is referred to as a proximal end side (near side) A2 of the clip unit 100.

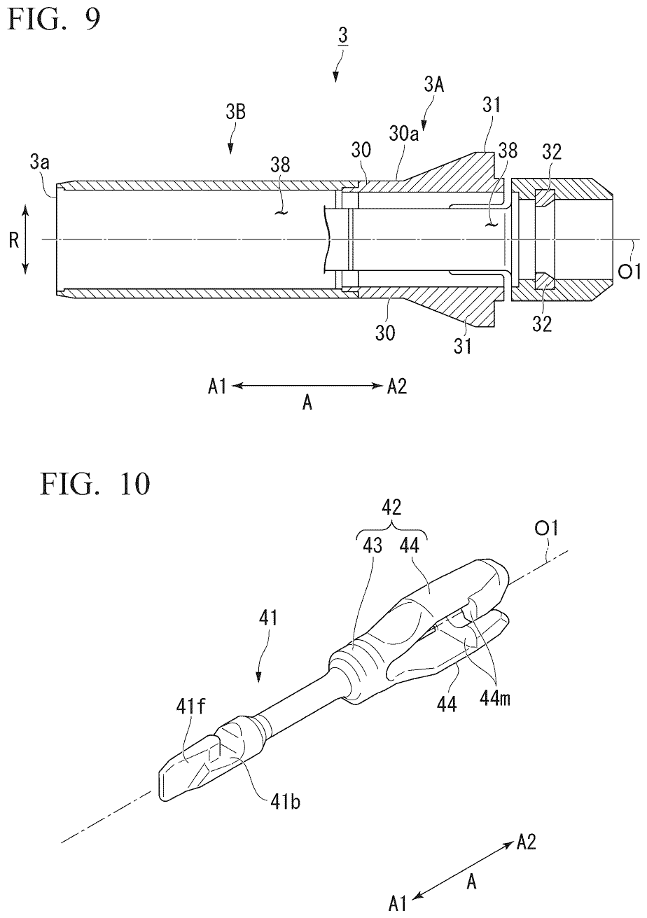

The clip 2 is formed by bending a metal plate material at its central portion. The clip 2 includes one pair of arms 21 that can open and close toward the distal end side A1 and a proximal end portion 28 that connects the pair of arms 21. The proximal end side A2 of the clip 2 is inserted into an internal space 38 of the pressing member 3 (see FIG. 9).

The pair of arms 21 have a first arm 211 and a second arm 212. The first arm 211 and the second arm 212 are arranged on both sides with a central axis line O1 of the clip unit 100 in the longitudinal direction A interposed therebetween. The clip 2 may have three or more arms.

The first arm 211 has a first claw 261, a grasping portion 23 having a flat plate shape, a sliding portion 24, and an engaging portion 25 from the distal end side A1 toward the proximal end side A2.

The second arm 212 has a second claw 262, a grasping portion 23 having a flat plate shape, a sliding portion 24, and an engaging portion 25 from the distal end side A1 toward the proximal end side A2.

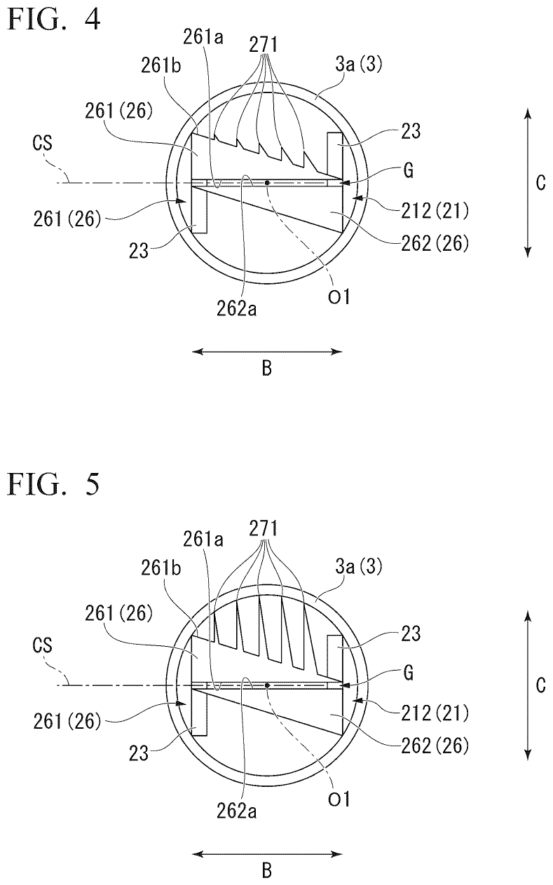

FIG. 4 is a diagram of one pair of arms 21 in a closed state that is seen in a longitudinal direction A.

Here, when one pair of arms 21 are in the closed state, a direction in which the first claw 261 and the second claw 262 oppose each other is referred to also as “opposing direction C”. In addition, a plane that passes through the central axis line O1 and is horizontal to an opening/closing direction B is referred to also as “opposing plane CS”.

In this embodiment, the opposing direction C is orthogonal to the opening/closing direction B of the pair of arms 21. However, the opposing direction C may not be configured to be orthogonal to the opening/closing direction B and may be a direction that intersects with the opening/closing direction B. In this embodiment, the longitudinal direction A is orthogonal to the opening/closing direction B of the pair of arms 21. However, the longitudinal direction A may not be configured to be orthogonal to the opening/closing direction B and may be a direction that intersects with the opening/closing direction B.

The first claw 261 is a member that can pierce a mucosa disposed at the distal end of the first arm 211 and extends to the inner side (the closing side) in the opening/closing direction B. The first claw 261 is formed by bending the distal end of the first arm 211 to the inner side in the opening/closing direction B. In other words, a bending portion is formed between the first arm 211 and the first claw 261. In the clip 2 in the closed state, the first arm 211 extends in the longitudinal direction A, and the first claw 261 extends in the opening/closing direction B. The first claw 261 is formed in a tapered shape that narrows toward the inner side in the opening/closing direction B. In this embodiment, the first claw 261 is formed in the shape of a right triangle when seen in the longitudinal direction A. The first claw 261 does not need to be formed in the shape of a right triangle when seen in the longitudinal direction A and, for example, may be formed in the shape of a triangle.

The first claw 261 has a plurality of first anchors 271 on an outer face (first outer face) 261b located on the outer side in the opposing direction C (on the side opposite to the second claw 262). The plurality of first anchors 271 extend from the first claw 261 toward the outer side in the opposing direction C and may be capable of piercing the mucosa. More specifically, the plurality of first anchors 271 are formed in a tapered shape that narrows toward the outer side in the opposing direction C.

The outer face 261b of the first claw 261 is inclined with respect to the opening/closing direction B and approaches an opposing face CS toward the inner side (closing side) in the opening/closing direction B.

The plurality of first anchors 271 are arranged in the opening/closing direction B on the outer face 261b of the first claw 261. The protrusion length of the plurality of first anchors 271 from the outer face 261b increases toward the inner side (closing side) in the opening/closing direction B.

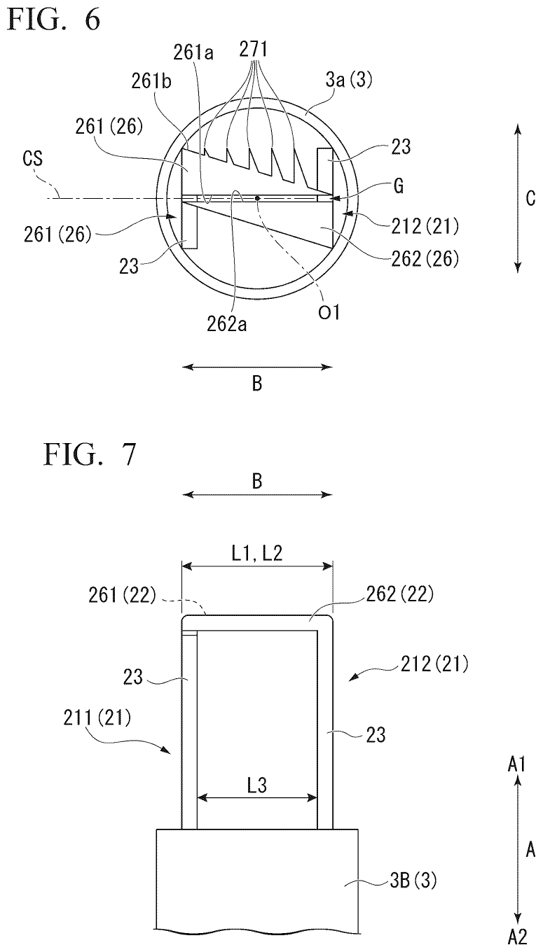

FIGS. 5 and 6 are diagrams illustrating modified examples of the first anchors 271.

The protrusion length of the plurality of first anchors 271 protruding from the outer face 261b may be uniform. As illustrated in FIG. 5, the protrusion length of the plurality of first anchors 271 may extend up to a length that fits within the lumen of the pressing member 3. In addition, as illustrated in FIG. 6, the protrusion length of the plurality of first anchors 271 may extend to the end of the grasping portion 23 in the opposing direction C.

The second claw 262 is a member that can pierce a mucosa disposed at the distal end of the second arm 212 and extends to the inner side (the closing side) in the opening/closing direction B. The second claw 262 is formed by bending the distal end of the second arm 212 to the inner side in the opening/closing direction B. In other words, a bending portion is formed between the second arm 212 and the second claw 262. In the clip 2 in the closed state, the second arm 212 extends in the longitudinal direction A, and the second claw 262 extends in the opening/closing direction B. The second arm 212 is formed in a tapered shape that narrows toward the inner side in the opening/closing direction B. In this embodiment, the second claw 262 is formed in the shape of a right triangle when seen in the longitudinal direction A. In addition, the second claw 262 does not need to be formed in the shape of a right triangle when seen in the longitudinal direction A and, for example, may be formed in the shape of a triangle.

The first claw 261 of the first arm 211 and the second claw 262 of the second arm 212 are formed in shapes that are asymmetric with respect to the central axis line O1. For this reason, when attaching the clip 2 to the connecting member 4, a user can easily perceive an orientation in which the clip 2 is to be attached to the connecting member 4. In addition, the first claw 261 of the first arm 211 and the second claw 262 of the second arm 212 may be formed in shapes that are symmetrical with respect to the central axis line O1.

FIG. 7 is a diagram of one pair of the arms 21 in the closed state that is seen in the opposing direction C.

In the opening/closing direction B, the length L1 of the first claw 261 is greater than the length L3 between the first arm 211 and the second arm 212 at the time when the pair of arms 21 are in the closed state. Further, in the opening/closing direction B, the length L2 of the second claw 262 is greater than a length L3 between the first arm 211 and the second arm 212 at the time when the pair of arms 21 are in the closed state.

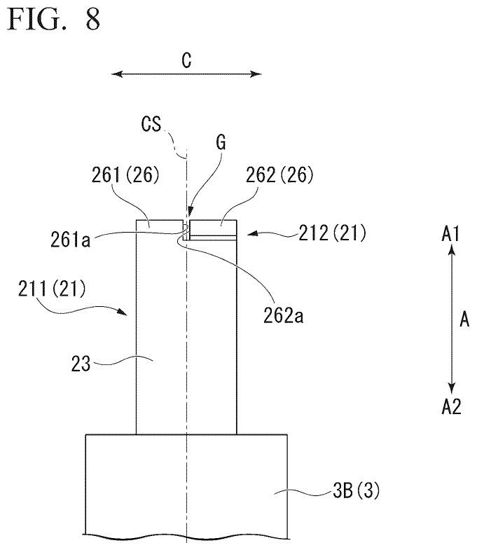

FIG. 8 is a diagram of one pair of the arms 21 in the closed state that is seen in the opening/closing direction B.

The first claw 261 and the second claw 262 are members that grasp a biological tissue. When the pair of the arms 21 are in the closed state, the first claw 261 and the second claw 262 are not in contact with each other. When the pair of the arms 21 are in the closed state, there is a gap G between the first claw 261 and the second claw 262 in the opposing direction C in which the first claw 261 and the second claw 262 face each other.

More specifically, when the pair of arms 21 are in the closed state, a first inner face 261a of the first claw 261 on the inner side in the opposing direction C and a second inner face 262a of the second claw 262 on the inner side in the opposing direction C are arranged to face each other with an opposing face CS, which passes through the central axis line O1 and is horizontal in the opening/closing direction B, interposed therebetween. When the pair of arms 21 are in the closed state, the first inner face 261a of the first claw 261 and the second inner face 262a of the second claw 262 are parallel to each other.

Furthermore, when the pair of the arms 21 are in the closed state, the positions of the first claw 261 and the second claw 262 in the longitudinal direction A are the same.

In the following description, when no distinction is necessary between the first claw 261 and the second claw 262, the first claw 261 and the second claw 262 are referred to also as “claw 26”.

The sliding portion 24 is a part that elastically deforms when the pair of the arms 21 are drawn into the pressing member 3.

The engaging portion 25 is a member that can engage with a fixing member 32 of the pressing member 3. The distal end side A1 of the engaging portion 25 is formed as an oblique face having an acute angle with respect to the longitudinal direction A, and the proximal end side A2 is formed as an oblique face having an obtuse angle with respect to the longitudinal direction A.

The proximal end portion 28 connects the first arm 211 and the second arm 212 to each other. The first arm 211 and the second arm 212, which are connected by the proximal end portion 28, are provided so as to be openable and closable toward the distal end side A1. The proximal end portion 28 is bent in the shape of “U” and is connected to a hook 41f of the connecting member 4. The proximal end portion 28 is biased such that the pair of arms 21 are in the open state. For this reason, the pair of arms 21 of the clip 2 have a self-expanding force in the opening/closing direction B.

As illustrated in FIG. 3, the proximal end portion 28 and the hook 41f of the connecting member 4 are connected by inserting the hook 41f of the connecting member 4 into the proximal end portion 28 formed in the shape of “U”.

FIG. 9 is a cross-sectional view of the pressing member 3 in the longitudinal direction A.

The pressing member (tube-shaped member) 3 is a cylinder-shaped member capable of storing at least a part of the clip 2. The pressing member 3 has an internal space 38 in which the clip 2 can advance and retract in the longitudinal direction A. The pressing member 3 can fix the clip 2, which has been drawn into the internal space 38, in the closed state. The pressing member 3 includes a pressing tube 3A provided on the proximal end side A2 and a pressing pipe 3B provided on the distal end side A1.

The pressing tube (a second tube-shaped member) 3A includes a pressing tube main body 30 formed in a cylinder shape, a protruding/recessed wing 31, and a fixing member 32.

The pressing tube main body 30 is formed by performing injection molding of a material that is softer than the clip 2, for example, a thermoplastic resin having proper elasticity such as polyphthalamide (PPA), polyamide (PA), polyether ether ketone (PEEK), or liquid crystal polymer (LCP). In addition, the pressing tube main body 30 may be formed of metal instead of a thermoplastic resin.

The protruding/recessed wings 31 are one pair of convex portions that protrude from an outer peripheral face 30a of the pressing tube main body 30. The protruding/recessed wings 31 are disposed on both sides with the central axis line O1 interposed therebetween. The protruding/recessed wings 31 have a protruding state in which they protrude to the outer side in the radial direction R from the outer peripheral face 30a as their basic orientation. When a force is applied from the outer side toward the inner side in the radial direction R, the protruding/recessed wings 31 enter a recessed state in which they are pushed inward relative to the outer peripheral face 30a. When the force described above is released, the protruding/recessed wings 31 return from the recessed state to the protruding state.

The fixing member 32 is a ring-shaped member disposed in the internal space 38 of the pressing tube 3A. The fixing member 32 is made of metal. In addition, the fixing member 32 only needs to be formed to be harder than the pressing tube main body 30 and, for example, may be made of a thermoplastic resin instead of metal.

The fixing member 32 is arranged such that the central axis of the fixing member 32 coincides with the central axis line O1. The fixing member 32 is arranged on the proximal end side A2 relative to the protruding/recessed wings 31. The fixing member 32 is, for example, incorporated into the pressing tube main body 30 through insert molding. The fixing member 32 is arranged at a position protruding from the inner peripheral face of the pressing tube main body 30 to the inner side in the radial direction R.

The pressing pipe (a first tube-shaped member) 3B is a cylindrical member made of metal. The pressing pipe 3B is press-fitted into the distal end portion of the pressing tube 3A. The pressing tube 3A and the pressing pipe 3B may be connected through thermal welding, adhesion, or screw fastening.

FIG. 10 is a perspective view of the connecting member 4.

The connecting member 4 is detachably connected to the proximal end portion 28 of the clip 2. In addition, the connecting member 4 is detachably connected to the arrowhead hook portion 231 that is inserted into and passes through the sheath 220. In other words, the connecting member 4 connects the clip 2 and the arrowhead hook portion 231 to each other. The connecting member 4 includes an insertion portion 41 that is inserted into the internal space 38 of the pressing member 3 and a connecting portion 42 provided at the proximal end of the insertion portion 41.

The insertion portion 41 includes a hook 41f provided in its distal end portion and a breaking portion 41b provided on the proximal end side A2 of the hook 41f. The hook 41f is a hook that extends in a direction perpendicular to the central axis line O1 and is formed in an approximately cylindrical rod shape. The proximal end portion 28 of the clip 2 is hooked onto the hook 41f.

The breaking portion 41b is broken when a tensile breaking force according to tension of, for example, 20 N to 90 N is applied to the hook 41f in accordance with the proximal end portion 28 being pulled to the proximal end side A2. The breaking portion 41b may have a mechanism that releases the connection between the proximal end portion 28 of the clip 2 and the hook 41f of the connecting member 4. For example, the breaking portion 41b may be a mechanism that releases the connection between the proximal end portion 28 and the hook 41f by deforming (plastically or elastically) without being actually broken.

The connecting portion 42 is an engaging portion which the arrowhead hook portion 231 of the clip introduction device 200 is engaged with (connected to). The connecting portion 42 includes a connecting portion main body 43 and an elastic arm portion 44.

The elastic arm portion 44 is disposed at the proximal end of the connecting portion main body 43 and branches into a bifurcated shape. The elastic arm portion 44 is elastically deformable relative to the connecting portion main body 43 and can be opened and closed with respect to the connecting portion main body 43. A notch portion 44m, which grasps and houses the engaging portion 231a of the arrowhead hook portion 231, is formed in the elastic arm portion 44. The notch portion 44m is formed in a shape that is brought into close contact with the outer peripheral face of the engaging portion 231a of the arrowhead hook portion 231.

### Operation and Action of Clip Unit 100

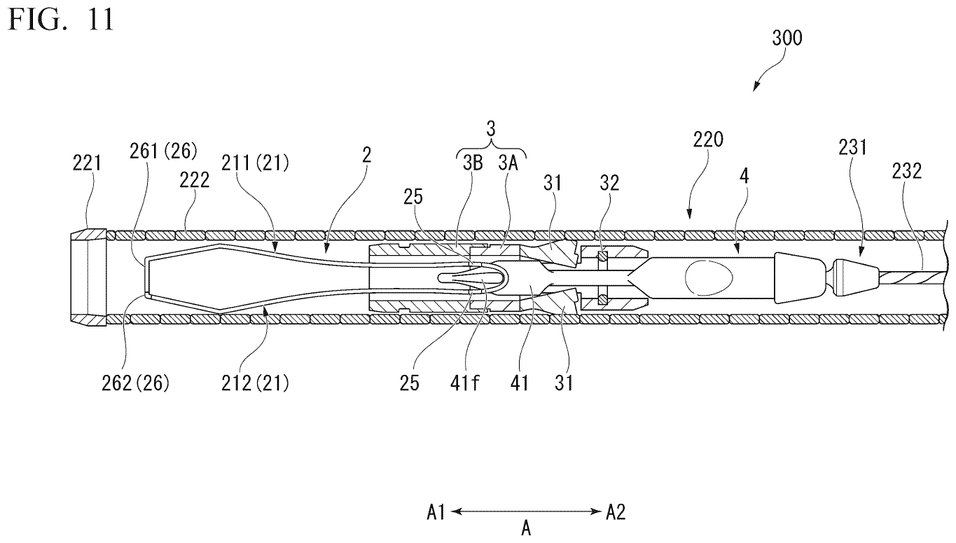

Next, operations and actions of the clip unit 100 are described with reference to FIGS. 11 to 21.

FIG. 11 is a diagram illustrating the clip unit 100 loaded into the clip introduction device 200.

The clip unit 100 is loaded into the clip introduction device 200 using a cartridge and the like. The connecting member 4 of the clip unit 100 loaded in the clip introduction device 200 is connected to the arrowhead hook portion 231 that is inserted into and passes through the inside of the sheath 220. The protruding/recessed wing 31 is pressed by the inner peripheral face of the sheath 220 to be in a recessed state.

One pair of the arms 21 loaded into the clip unit 100 are in the state of being pressed by the inner peripheral face of the sheath 220. The engaging portion 25 is positioned on the distal end side A1 relative to the fixing member 32, and the pair of the arms 21 are not locked in the closed state.

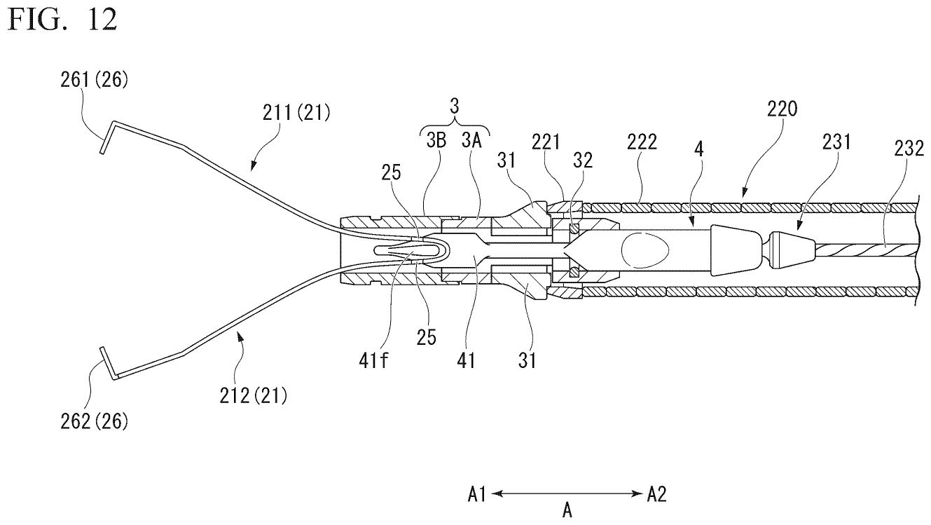

FIG. 12 is a diagram illustrating the clip unit 100 introduced into the inside of the body.

An operator introduces the clip unit 100 that has been loaded into the sheath 220 into the body through the channel of the endoscope. Next, by advancing the slider 242 along the operation unit main body 241, the operator moves the arrowhead hook portion 231 forward. The operator advances the clip unit 100 until the protruding/recessed wing 31 comes out of the sheath 220. When the protruding/recessed wing 31 comes out of the sheath 220, it returns from the recessed state to the protruding state that is its basic orientation.

When the distal end side of the pair of arms 21 protrudes from the sheath 220, the clip 2 returns to the open state while moving to the distal end side with respect to the pressing member 3 by using a self-expanding force of the pair of arms 21 as a restoring force. Even in a case in which the pair of arms 21 have returned to the open state and protrude the most from the pressing member 3, the engaging portion 25 is arranged in an internal area of the pressing member 3.

By moving the endoscope and the sheath 220, the operator brings the clip 2 of the clip unit 100 close to the defective portion D that is a treatment area. In the following description, mucosae that form edges surrounding the defective portion D and are mucosae arranged to face each other with the defective portion D interposed therebetween are referred to as a “first mucosa M1” and a “second mucosa M2”.

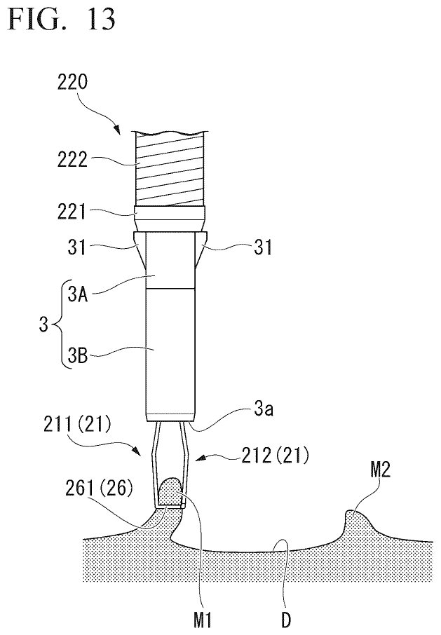

FIG. 13 is a diagram illustrating the clip unit 100 that grasps the first mucosa M1.

The operator grasps the first mucosa M1 with one pair of arms 21. The operator pierces the first mucosa M1 with the first claw 261 and the second claw 262.

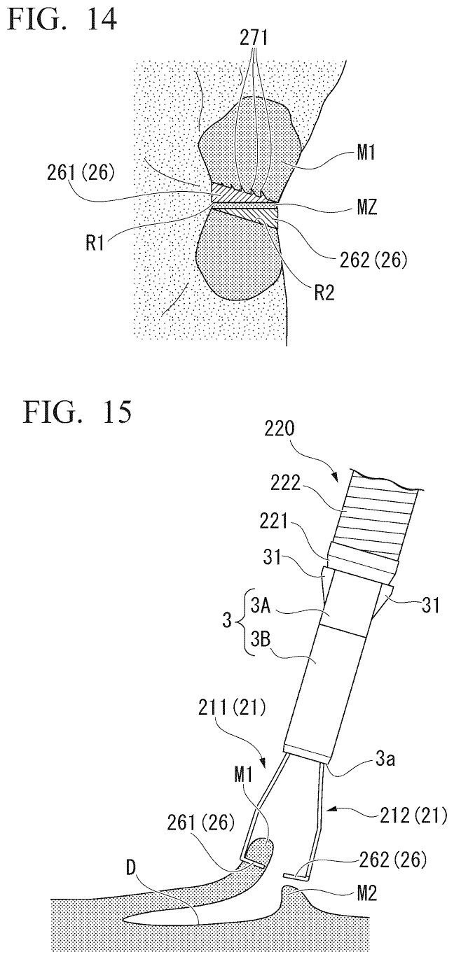

FIG. 14 is a diagram illustrating the first mucosa M1 grasped by one pair of arms 21.

When the first claw 261 and the second claw 262 pierce the first mucosa M1, a mucosal tissue MZ is arranged in a gap G formed between the first claw 261 and the second claw 262. For this reason, a hole area R1 formed by the first claw 261 piercing the first mucosa M1 and a hole area R2 formed by the second claw 262 piercing the first mucosa M1 do not communicate with each other.

The operator draws the first mucosa M1, which is grasped by the pair of arms 21, to the second mucosa M2 side. Since the first mucosa M1 is firmly grasped by the first claw 261 and the second claw 262, the operator can easily draw the first mucosa M1 to the second mucosa M2 side.

FIG. 15 is a diagram illustrating the clip unit 100 after the second claw 262 has been pulled out from the first mucosa M1. The operator pulls out the second claw 262 from the first mucosa M1 in order to grasp the second mucosa M2 with the second arm 212.

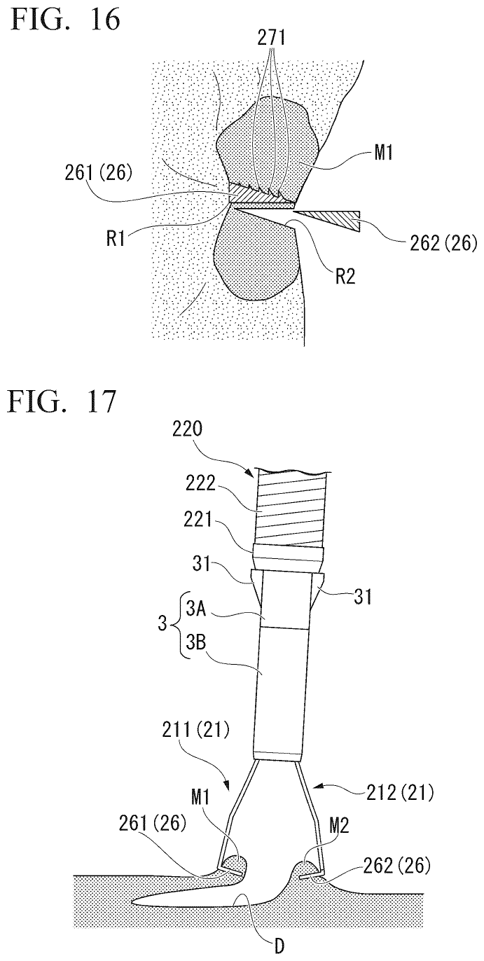

FIG. 16 is a diagram illustrating the first mucosa M1 after the second claw 262 has been pulled out.

Since no anchor is provided in the second claw 262, it is easy for the second claw 262 to be pulled out from the first mucosa M1. On the other hand, since a first anchor 271 is provided in the first claw 261, it is difficult for the first claw 261 to be pulled out from the first mucosa M1. Furthermore, since the hole area R1 and the hole area R2 do not communicate with each other, even after the second claw 262 has been pulled out from the first mucosa M1, the hole area R1 is not connected to the hole area R2, and the hole area R1 does not expand. Accordingly, it is difficult for the first claw 261 to be pulled out from the first mucosa M1. As a result, the operator can easily pull out the second claw 262 from the first mucosa M1 in a state in which the first claw 261 pierces into the first mucosa M1.

FIG. 17 is a diagram illustrating the clip unit 100 that grasps the second mucosa M2.

An operator grasps the second mucosa M2 with the second arm 212. The operator pierces the second mucosa M2 with the second claw 262. As a result, the first mucosa M1 is grasped by the first arm 211, and the second mucosa M2 is grasped by the second arm 212.

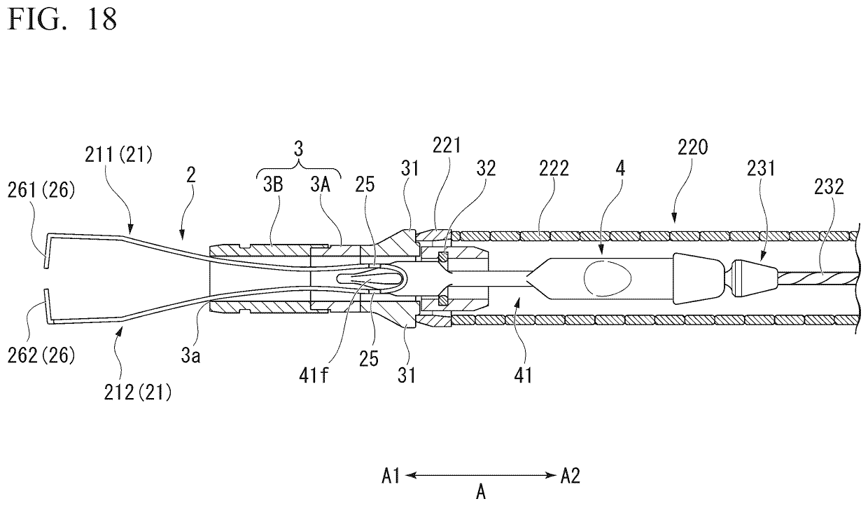

FIG. 18 is a diagram illustrating the clip unit 100 with one pair of arms 21 closed.

An operator retracts the arrowhead hook portion 231 by retracting the slider 242 along the operation unit main body 241. The connecting member 4, which is connected to the arrowhead hook portion 231, pulls the clip 2. The pair of arms 21, having a self-opening force, are pulled to the proximal end side A2, thereby pressing a distal end opening 3a of the pressing member 3 to the proximal end side A2. Since the protruding/recessed wing 31, which is in its protruded state, is engaged with the sheath 220, it is not drawn into the inside of the sheath 220. For this reason, the clip 2, which is pulled by the connecting member 4, is drawn into the pressing member 3.

By pulling the proximal end portion 28 of the clip 2 to the proximal end side A2 of the pressing member 3 in accordance with the connecting member 4, the pair of arms 21 are drawn into the pressing member 3, and the pair of arms 21 are gradually closed.

When the pulling force applied to the proximal end portion 28 is released in this state, the clip 2 returns to the open state while moving to the distal end side A1 using the self-expanding force of the pair of arms 21 as a restoration force. The operator can return the pair of arms 21 to the open state and re-grasp the tissue.

FIG. 19 is a diagram illustrating the clip unit 100 in which the clip 2 is locked.

When the proximal end portion 28 is further pulled to the proximal end side A2 of the pressing member 3, the engaging portion 25 is further drawn to the proximal end side A2 relative to the fixing member 32. Since the proximal end side A2 of the engaging portion 25 is formed on an oblique face having an obtuse angle, the engaging portion 25 can be easily drawn to the proximal end side relative to the fixing member 32. On the other hand, since the distal end side A1 of the engaging portion 25 is formed on a slope having an acute angle, when the engaging portion 25 is drawn to the proximal end side A2 relative to the fixing member 32, the engaging portion 25 and the fixing member 32 are engaged with each other. As a result, the movement of the clip 2 to the distal end side A1 relative to the pressing member 3 is regulated, and the pair of arms 21 are locked in the closed state. When the pair of arms 21 are locked in the closed state, the pair of arms 21 cannot return to the open state.

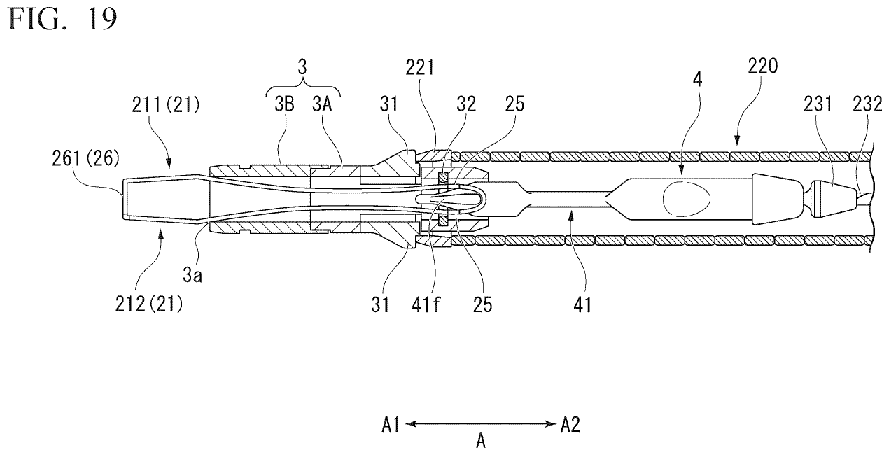

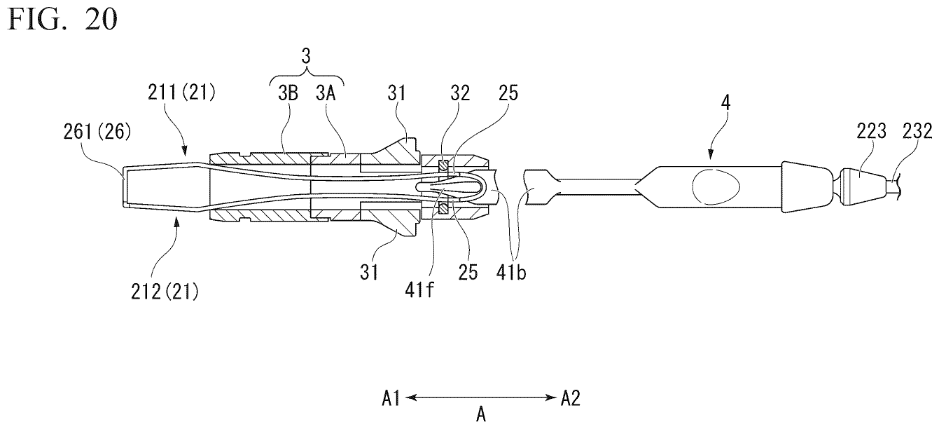

FIG. 20 is a diagram illustrating the clip unit 100 in which the clip 2 has been separated.

An operator further pulls the clip 2. A tensile breaking force of, for example, 20 N (newtons) to 90 N according to pulling is applied to the hook 41f, and the breaking portion 41b is broken. The breaking strength of the breaking portion 41b is lower than that of the connecting portion main body 43. For this reason, not the connecting portion main body 43 but the breaking portion 41b is broken.

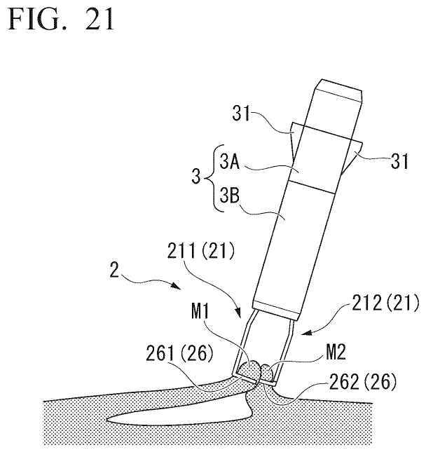

FIG. 21 is a diagram illustrating the clip unit 100 after breakage.

The operator retracts the sheath 220 and places the clip 2 that is in the state of ligating the tissue inside the body.

The operator reloads a new clip unit 100 into the clip introduction device 200 using a cartridge or the like.

According to the clip delivery device 300 of this embodiment, the treatment of closing and suturing a defective portion D can be appropriately performed.

As above, while the first embodiment of the present invention has been described in detail with reference to the drawings, a specific configuration is not limited to this embodiment and includes a change in the design and the like in a range not departing from the gist of the present invention. In addition, components represented in the embodiment described above and modified examples represented below can be appropriately combined and configured.

### Modified Example 1-1

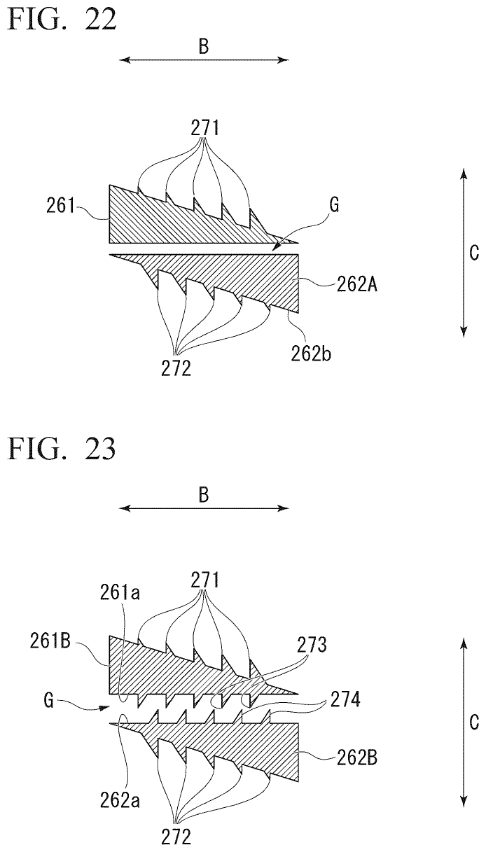

FIG. 22 is a diagram illustrating a second claw 262A that is a modified example of the second claw 262.

The second claw 262A has a plurality of second anchors 272 on a second outer face 262b, which is located on the outer side (a side opposite to the first claw 261 side) in the opposing direction C. Compared with the second claw 262, it is difficult for the second claw 262A to be pulled out from the mucosa.

### Modified Example 1-2

FIG. 23 is a diagram illustrating a first claw 261B and a second claw 262B that are modified examples of the first claw 261 and the second claw 262A. The first claw 261B has a plurality of third anchors 273 on a first inner face 261a. The plurality of third anchors 273 extend from the first claw 261B to the inner side in the opposing direction C and can pierce the mucosa. The second claw 262B has a plurality of fourth anchors 274 on a second inner face 262a. The plurality of fourth anchors 274 extend from the second claw 262B to an inner side in the opposing direction C and can pierce the mucosa.

### Modified Example 1-3

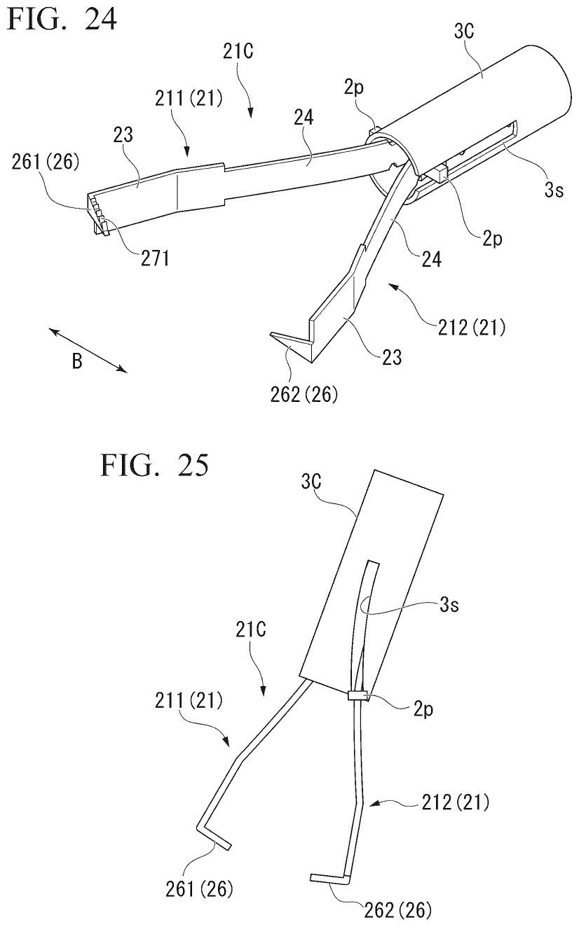

FIGS. 24 and 25 are diagrams illustrating a pressing member 3C that is a modified example of the pressing member 3 and one pair of arms 21C that is a modified example of the one pair of arms 21. The pair of arms 21C further have convex portions 2p that protrude to the outer side in the opening/closing direction B in the sliding portion 24. The pressing member 3C has a slit 3s that is engaged with the convex portions 2p. The slit 3s is formed in a linear shape along the longitudinal direction A. For this reason, the pair of arms 21C can advance and retract without tilting relative to the longitudinal direction A. That is, the clip 2 can maintain a gap G between the first claw 261 and the second claw 262 both in the open state and in the closed state. In other words, even when the clip 2 is either in the open state or in the closed state, the positions of the first claw 261 and the second claw 262 in the opposing direction C are different. As illustrated in FIG. 25, the slit 3s may be formed in a curved shape. In this case, the pair of arms 21C have convex portions 2p that protrude to the outer side in the opposing direction C in the sliding portion 24. The convex portions 2p has a first portion and a second portion. The first portion has a width smaller than that of the slit 3s and slides inside of the slit 3s. The second portion is arranged on the outer side of the pressing member 3C and has a width greater than that of the slit 3s. In accordance with the second portion of the convex portion 2p coming into contact with the pressing member 3, movement of the first claw 261 and the second claw 262 in a direction in which they approach each other is regulated in the opposing direction C. For this reason, the pair of arms 21C can advance and retract without tilting with respect to the longitudinal direction A. That is, the clip 2 can maintain the gap G between the first claw 261 and the second claw 262 both in the open state and in the closed state. In other words, even when the clip 2 is either in the open state or in the closed state, the positions of the first claw 261 and the second claw 262 in the opposing direction C are different.

### Modified Example 1-4

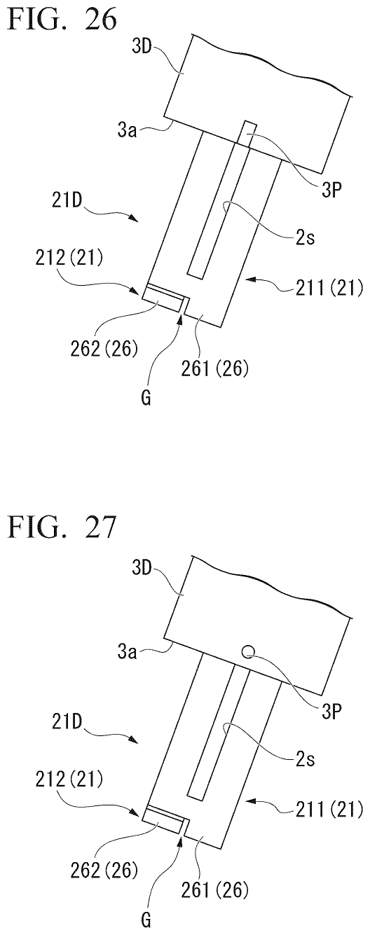

FIGS. 26 and 27 are diagrams illustrating a pressing member 3D that is a modified example of the pressing member 3 and one pair of arms 21D that is a modified example of the pair of arms 21. The pressing member 3D further has a convex portion 3p that protrudes to the inner side at the distal end opening 3a. The pair of arms 21D have slits 2s that are engaged with the convex portions 3p. The slit 2s is formed in a linear shape in the longitudinal direction A. For this reason, the pair of arms 21D can advance and retract without tilting relative to the longitudinal direction A. That is, the clip 2 can maintain a gap G between the first claw 261 and the second claw 262 both in the open state and in the closed state. In other words, even when the clip 2 is either in the open state or in the closed state, the positions of the first claw 261 and the second claw 262 in the opposing direction C are different. As illustrated in FIG. 27, the convex portion 3p may be a pin that connects opposing inner faces of the distal end opening 3a.

### Second Embodiment

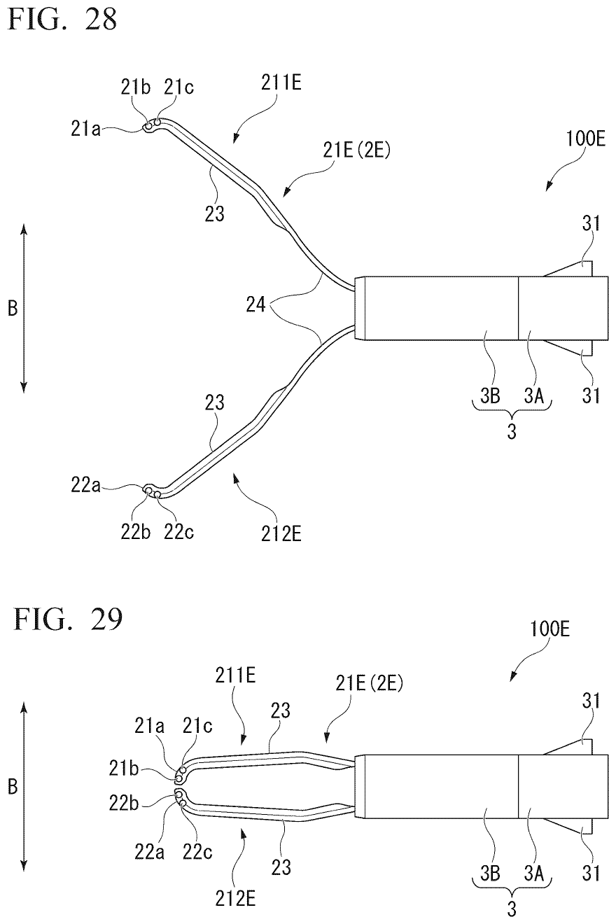

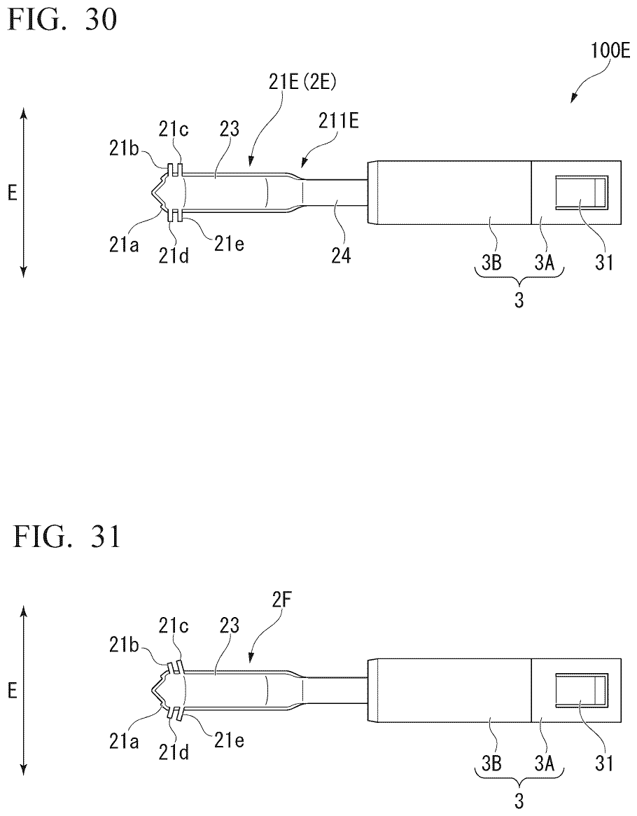

A second embodiment of the present invention is described with reference to FIGS. 28 to 30. In the following description, the same reference signs are assigned to the components common to those described above, and a duplicate description thereof is omitted.

FIGS. 28 to 30 are diagrams illustrating a clip unit 100E according to a second embodiment.

The clip unit 100E includes a clip 2E, a pressing member 3, and a connecting member 4. The connecting member 4 is not illustrated in FIGS. 28 to 30.

The clip 2E is formed by bending a metal plate material at its central portion. The clip 2E includes one pair of arms 21E that can open and close toward the distal end side A1 and a proximal end portion 28 that connects the pair of arms 21E. The proximal end side A2 of the clip 2E is inserted into an internal space 38 of the pressing member 3.

The pair of arms 21E have a first arm 211E and a second arm 212E. The first arm 211E and the second arm 212E are arranged on both sides with a central axis line O1 of the clip unit 100E in the longitudinal direction A interposed therebetween. The clip 2E may have three or more arms.

The first arm 211E has a first claw 21a, a grasping portion 23 having a flat plate shape, a sliding portion 24, and an engaging portion 25 from the distal end side A1 toward the proximal end side A2.

The second arm 212E has a second claw 22a, a grasping portion 23 having a flat plate shape, a sliding portion 24, and an engaging portion 25 from the distal end side A1 toward the proximal end side A2.

As illustrated in FIG. 29, when the pair of arms 21E are in the closed state, the first claw 21a and the second claw 22a face each other in the opening/closing direction B. In other words, in this embodiment, when the pair of arms 21E are in the closed state, the opposing direction C in which the first claw 21a and the second claw 22a face each other coincides with the opening/closing direction B. In addition, when the pair of arms 21E are in the closed state, the first claw 21a and the second claw 22a may be in contact with each other.

The first claw 21a has a right side face and a left side face in a lateral direction E that is orthogonal to the opening/closing direction B. On the right side face of the first claw 21a, right-side anchors 21b and 21c protruding in the lateral direction E are provided. On the left side face of the first claw 21a, left-side anchors 21d and 21e protruding in the lateral direction E are provided.

The second claw 22a has a right side face and a left side face in the lateral direction E that are orthogonal to the opening/closing direction B. On the right side face of the second claw 22a, right-side anchors 22b and 22c protruding in the lateral direction E are provided. On the left side face of the second claw 22a, left-side anchors 22d and 22e (not illustrated in the drawing) protruding in the lateral direction E are provided.

When approaching a defective portion D obliquely, the right-side anchors 21b and 21c of the first arm 211E and the right-side anchors 22b and 22c of the second arm 212E come into contact with the tissue before the first claw 21a and the second claw 22a. For this reason, the right-side anchors 21b, 21c, 22b, and 22c generate a large frictional force against the tissue, thereby preventing slippage from the tissue. This similarly applies also to the left-side anchors 21d and 21e of the first arm 211E and the left-side anchors 22d and 22e of the second arm 212E.

According to the clip unit 100E of this embodiment, the treatment of closing and suturing a defective portion D can be appropriately performed.

As above, while the second embodiment of the present invention has been described in detail with reference to the drawings, a specific configuration is not limited to this embodiment and includes a change in the design and the like in a range not departing from the gist of the present invention. In addition, components represented in the embodiment described above and modified examples represented below can be appropriately combined and configured.

### Modified Example 2-1

FIG. 31 is a diagram illustrating a clip 2F that is a modified example of the clip 2E.

In the clip 2F, the right-side anchor 21c on the proximal end side A2 is longer than a right-side anchor 21b on the distal end side A1. For this reason, when the clip 2F approaches a defective portion D obliquely, both the right-side anchors 21b and 21c easily come into contact with a tissue. More specifically, when the clip 2F approaches the defective portion D obliquely such that the right side faces of the first claw 21a and the second claw 22a face the defective portion D, the right-side anchors 21b, 21c, 22b, and 22c are oriented in the direction of the defective portion D. At this time, since the right-side anchor 21c is longer than the right-side anchor 21b, both the right-side anchor 21b and the right-side anchor 21c easily come into contact with a tissue simultaneously. In accordance with this, a large frictional force is generated between the right-side anchors 21b, 21c, 22b, and 22c and the tissue, whereby the first claw 21a and the second claw 22a are suppressed from slipping off the tissue. This similarly applies also to the left-side anchors 21d, 21e, 22d, and 22e.

### Modified Example 2-2

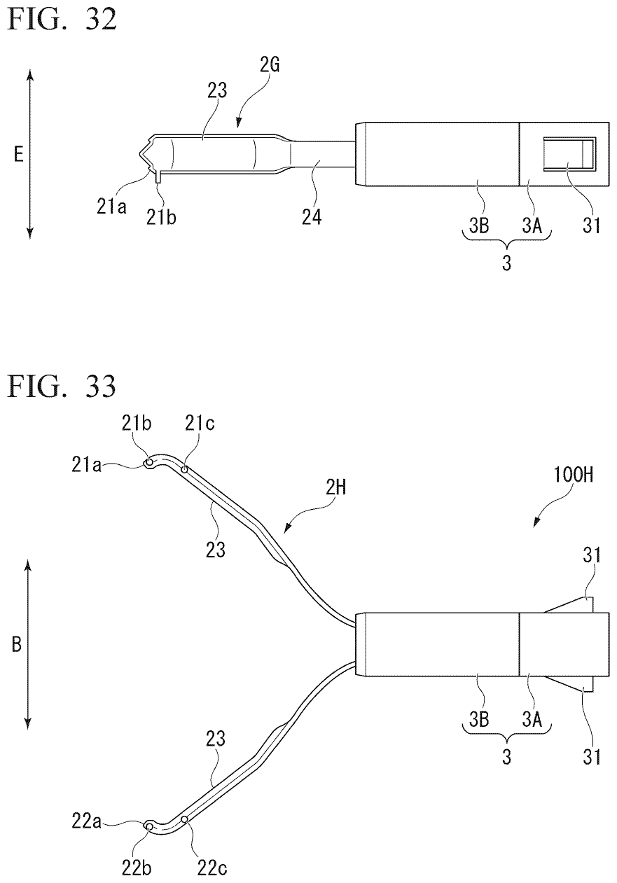

FIG. 32 is a diagram illustrating a clip 2G that is a modified example of the clip 2E.

In the embodiment described above, the number of anchors in one arm (for example, the right-side anchors 21b and 21c and the left-side anchors 21d and 21e) is four. However, the number of anchors is not limited to this. For example, as illustrated in FIG. 32, the number of anchors in one arm may be one or may be two or three.

### Modified Example 2-3

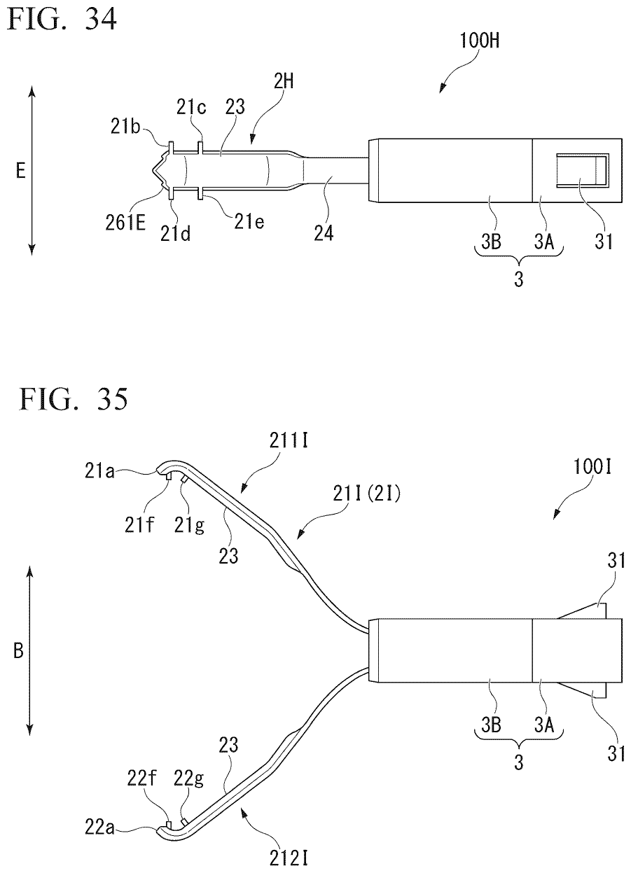

FIGS. 33 and 34 are diagrams illustrating a clip 2H that is a modified example of the clip 2E.

In the embodiment described above, the anchors (for example, the right-side anchors 21b and 21c and the left-side anchors 21d and 21e) are provided on the claw (for example, the first claw 21a). However, the positions at which the anchors are arranged are not limited thereto. As illustrated in FIGS. 33 and 34, at least some of the anchors may be provided on the grasping portion 23 of the proximal end side A2 of the claw.

### Third Embodiment

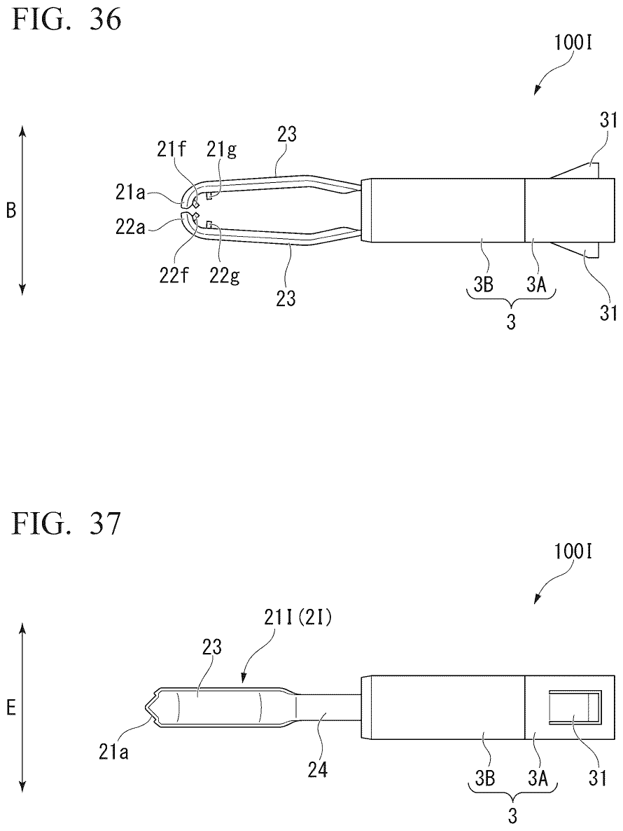

A third embodiment of the present invention is described with reference to FIGS. 35 to 37. In the following description, the same reference signs are assigned to the components common to those described above, and a duplicate description thereof is omitted.

FIGS. 35 to 37 are diagrams illustrating a clip unit 100I according to a third embodiment.

The clip unit 100I includes a clip 2I, a pressing member 3, and a connecting member 4. The connecting member 4 is not illustrated in FIGS. 35 to 37.

The clip 2I is formed by bending a metal plate material at its central portion. The clip 2I includes one pair of arms 21I that can open and close toward the distal end side A1 and a proximal end portion 28 that connects the pair of arms 21I. The proximal end side A2 of the clip 2I is inserted into an internal space 38 of the pressing member 3.

The pair of arms 21I have a first arm 211I and a second arm 212I. The first arm 211I and the second arm 212I are arranged on both sides with a central axis line O1 of the clip unit 100I in the longitudinal direction A interposed therebetween. The clip 2I may have three or more arms.

The first arm 211I has a first claw 21a, inner face anchors 21f and 21g, a grasping portion 23 having a flat plate shape, a sliding portion 24, and an engaging portion 25 from the distal end side A1 toward the proximal end side A2.

The second arm 212I has a second claw 22a, inner face anchors 22f and 22g, a grasping portion 23 having a flat plate shape, a sliding portion 24, and an engaging portion 25 from the distal end side A1 toward the proximal end side A2.

The inner face anchors 21f and 21g are anchors that are provided on inner faces of the distal end portions of the first claw 21a and the grasping portion 23 of the first arm 211I and protrude toward the closing side in the opening/closing direction B. The inner face anchors 21f and 21g are arranged in the longitudinal direction A, and the inner face anchor 21f is arranged closer to the distal end side A1 than the inner face anchor 21g.

The inner face anchors 22f and 22g are anchors that are provided on inner faces of the distal end portions of the second claw 22a and the grasping portion 23 of the second arm 212I and protrude toward the closing side in the opening/closing direction B. The inner face anchors 22f and 22g are arranged in the longitudinal direction A, and the inner face anchor 22f is arranged closer to the distal end side A1 than the inner face anchor 22g.

Even when the pair of arms 21I are in an open state, when the pair of arms 21I are pressed against a biological tissue, both the inner face anchors 21f and 21g (the inner face anchors 22f and 22g) come into contact with the tissue.

According to the clip unit 100I of this embodiment, the treatment of closing and suturing a defective portion D can be appropriately performed.

As above, while the third embodiment of the present invention has been described in detail with reference to the drawings, a specific configuration is not limited to this embodiment and includes a change in the design and the like in a range not departing from the gist of the present invention. In addition, components represented in the embodiment described above and modified examples represented below can be appropriately combined and configured.

### Modified Example 3-1

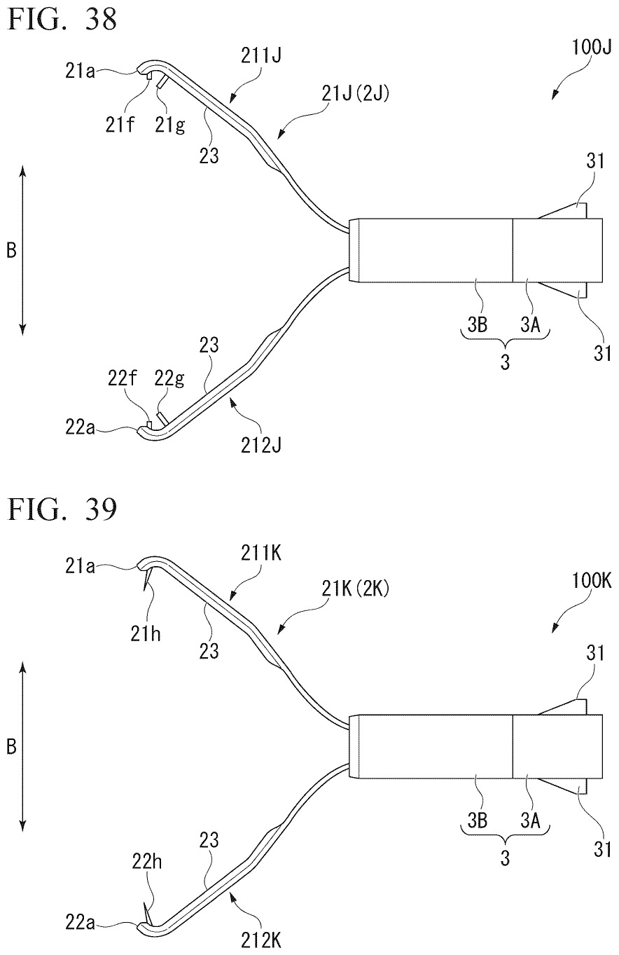

FIG. 38 is a diagram illustrating a clip 2J that is a modified example of the clip 2I.

In the clip 2J, the inner face anchor 21g on the proximal end side A2 is longer than the inner face anchor 21f on the distal end side A1. For this reason, when the clip 2J approaches a defective portion D obliquely and when it approaches a defective portion D from the front, both the inner face anchors 21f and 21g easily come into contact with a tissue. This similarly applies also to the inner face anchors 22f and 22g.

### Fourth Embodiment

A fourth embodiment of the present invention is described with reference to FIGS. 39 to 41. In the following description, the same reference signs are assigned to the components common to those described above, and a duplicate description thereof is omitted.

FIGS. 39 to 41 are diagrams illustrating a clip unit 100K according to a fourth embodiment.

The clip unit 100I includes a clip 2K, a pressing member 3, and a connecting member 4. The connecting member 4 is not illustrated in FIGS. 39 to 41.

The clip 2K is formed by bending a metal plate material at its central portion. The clip 2K includes one pair of arms 21K that can open and close toward the distal end side A1 and a proximal end portion 28 that connects the pair of arms 21K. The proximal end side A2 of the clip 2K is inserted into an internal space 38 of the pressing member 3.

The pair of arms 21K have a first arm 211K and a second arm 212K. The first arm 211K and the second arm 212K are arranged on both sides with a central axis line O1 of the clip unit 100K in the longitudinal direction A interposed therebetween. The clip 2K may have three or more arms.

The first arm 211K has a first claw 21a, an inner face anchor 21h, a grasping portion 23 having a flat plate shape, a sliding portion 24, and an engaging portion 25 from the distal end side A1 toward the proximal end side A2.

The second arm 212K has a second claw 22a, an inner face anchor 22h, a grasping portion 23 having a flat plate shape, a sliding portion 24, and an engaging portion 25 from the distal end side A1 toward the proximal end side A2.

The inner face anchor (needle) 21h is an anchor that is provided on the inner face of the first claw 21a of the first arm 211K and protrudes toward the closing side in the opening/closing direction B. The inner face anchor 21h is formed in a conical shape and has a distal end that is sharper than that of the inner face anchor 21f according to the third embodiment.

The inner face anchor (needle) 22h is an anchor that is provided on the inner face of the second claw 22a of the second arm 212K and protrudes toward the closing side in the opening/closing direction B. The inner face anchor 22h is formed in a conical shape and has a distal end that is sharper than that of the inner face anchor 22f according to the third embodiment.

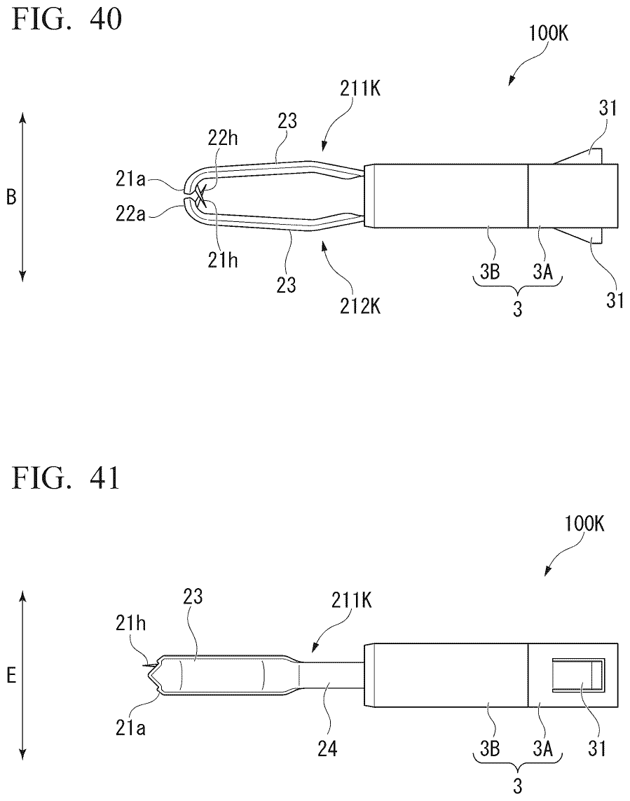

As illustrated in FIG. 40, when the pair of arms 21K are in the closed state, the inner face anchor 21h and the inner face anchor 22h intersect with each other. More specifically, when the pair of arms 21K are in the closed state, the position at which the inner face anchor 21h of the first arm 211K is arranged and the position at which the inner face anchor 22h of the second arm 212K is arranged slightly deviate from each other in the lateral direction E that is orthogonal to the opening/closing direction B, and the inner face anchor 21h and the inner face anchor 22h intersect with each other when viewed in the lateral direction E.

When the pair of arms 21K are pressed against a biological tissue, the inner face anchor 21h (the inner face anchor 22h) comes into contact with the tissue before the first claw 21a (the second claw 22a). The inner face anchor 21h (the inner face anchor 22h) penetrates the tissue. In accordance with this, a large frictional force is generated between the first claw 21a (the second claw 22a) and the tissue, whereby the first claw 21a (the second claw 22a) is suppressed from slipping off the tissue.

According to the clip unit 100K of this embodiment, the treatment of closing and suturing a defective portion D can be appropriately performed.

As above, while the fourth embodiment of the present invention has been described in detail with reference to the drawings, a specific configuration is not limited to this embodiment and includes a change in the design and the like in a range not departing from the gist of the present invention. In addition, components represented in the embodiment described above and modified examples represented below can be appropriately combined and configured.

### Modified Example 4-1

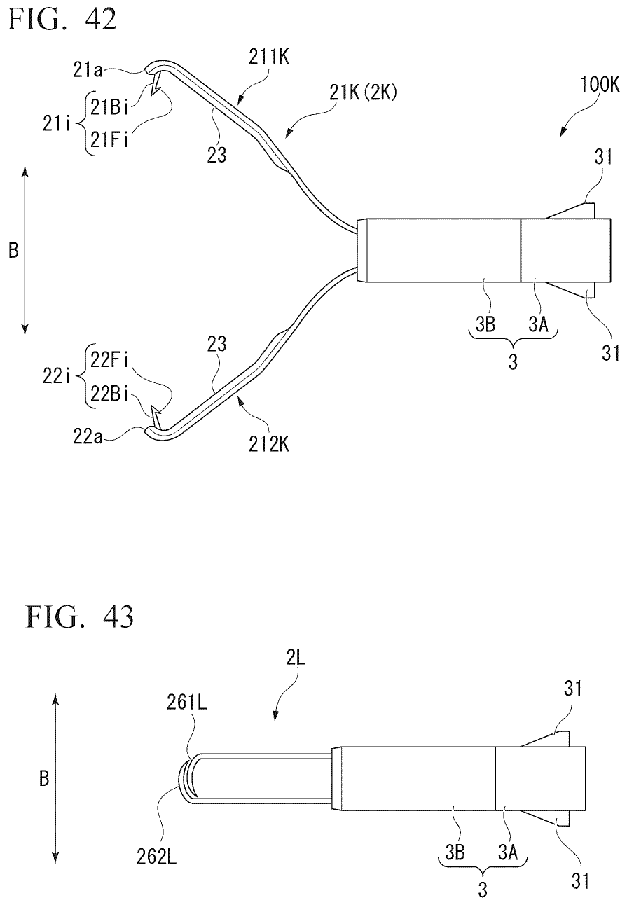

FIG. 42 is a diagram illustrating inner face anchors 21i and 22i that are modified examples of the inner face anchors 21h and 22h. The inner face anchor 21i has a hook 21Fi that extends from the distal end of the inner face anchor main body 21Bi toward the first arm 211K. An angle formed between the inner face anchor main body 21Bi and the hook 21Fi may be an acute angle. When the inner face anchor 21i penetrates the tissue, the hook 21Fi catches on the tissue. In accordance with this, the first claw 21a is suppressed from slipping off the tissue.

The inner face anchor 22i has a hook 22Fi that extends from the distal end of the inner face anchor main body 22Bi toward the second arm 212K. An angle formed between the inner face anchor main body 22Bi and the hook 22Fi may be an acute angle. When the inner face anchor 22i penetrates the tissue, the hook 22Fi catches on the tissue. In accordance with this, the second claw 22a is suppressed from slipping off the tissue.

### Modified Example A

FIG. 43 is a diagram illustrating a clip 2L that is a modified example of the clip 2.

A first claw 261L and a second claw 262L, which are provided at the distal end of the clip 2L, are formed in a curved shape that bends to the inner side in the opening/closing direction B. In the clip 2L that is in the closed state, the first claw 261L and the second claw 262L deviate from each other in the longitudinal direction A and are not engaged with each other. In the clip 2L that is in the open state, the first claw 261L and the second claw 262L are in contact with the tissue at approximately 90 degrees.

### Modified Example B

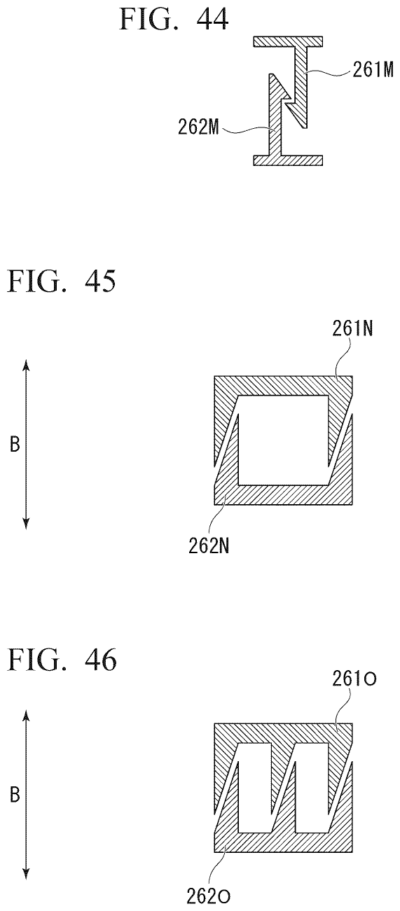

FIG. 44 is a diagram illustrating a first claw 261M and a second claw 262M that are modified examples of the first claw 261 and the second claw 262. The first claw 261M and the second claw 262M have locking mechanisms that perform locking at the time of engagement.

### Modified Example C

FIG. 45 is a diagram illustrating a first claw 261N and a second claw 262N that are modified examples of the first claw 261 and the second claw 262. Two first claws 261N are provided in a direction perpendicular to the opening/closing direction B. Two second claws 262N are provided in a direction perpendicular to the opening/closing direction B.

### Modified Example D

FIG. 46 is a diagram illustrating a first claw 261O and a second claw 262O that are modified examples of the first claw 261 and the second claw 262. Three first claws 261O are provided in a direction perpendicular to the opening/closing direction B. Three second claws 262O are provided in a direction perpendicular to the opening/closing direction B.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A clip unit comprising:
a clip having a first arm and a second arm that are capable of opening and closing; and
a tube-shaped member capable of storing at least a part of the clip,
wherein the first arm has a first claw that extends to a closing side in an opening/closing direction, and
wherein the first claw has a first anchor.

2. The clip unit according to claim 1, wherein the first claw is formed in a tapered shape narrowing toward the closing side in the opening/closing direction.

3. The clip unit according to claim 1, wherein the first anchor extends in a direction intersecting with the opening/closing direction.

4. The clip unit according to claim 3,
wherein the second arm has a second claw extending to a closing side in an opening/closing direction,
wherein the first claw and the second claw face each other in an opposing direction when the first arm and the second arm are in a closed state, and
wherein the first anchor extends from the first claw in the opposing direction.

5. The clip unit according to claim 4, wherein the first anchor extends to an outer side in the opposing direction.

6. The clip unit according to claim 1, wherein the first anchor is disposed on a side in a direction orthogonal to the opening/closing direction of the first claw.

7. The clip unit according to claim 1, wherein the first anchor extends from an inner side in the opening/closing direction of the first claw to the closing side in the opening/closing direction.

8. The clip unit according to claim 1, wherein a plurality of the first anchors is disposed.

9. The clip unit according to claim 1,
wherein the second arm has a second claw extending to a closing side in an opening/closing direction at,
wherein the first claw and the second claw face each other and form a gap in an opposing direction when the first arm and the second arm are in a closed state.

10. The clip unit according to claim 9,
wherein a first inner face on an inner side of the first claw in the opposing direction and a second inner face on an inner side of the second claw in the opposing direction are arranged to face each other when the first arm and the second arm are in the closed state, and
wherein the first inner face and the second inner face are flat.

11. The clip unit according to claim 10, wherein the first inner face and the second inner face are arranged in parallel with each other when the first arm and the second arm are in the closed state.

12. The clip unit according to claim 10, wherein the second claw has a second anchor extending to an outer side in the opposing direction.

13. The clip unit according to claim 9, further comprising a slit extending in a longitudinal direction of the tube-shaped member,
wherein movement of the first arm and the second arm in the opposing direction is regulated by the slit such that the gap is formed between the first claw and the second claw.

14. The clip unit according to claim 13,
wherein the slit is disposed in the tube-shaped member,
wherein the first arm and the second arm have convex portions that are engageable with the slit, and
wherein the convex portions extend to an outer side of the tube-shaped member in a radial direction.

15. The clip unit according to claim 13,
wherein the slit is disposed in the first arm and the second arm,
wherein the tube-shaped member has a convex portion that is engageable with the slit, and
wherein the convex portion extends to an inner side of the tube-shaped member in a radial direction.

16. The clip unit according to claim 1, wherein a length of the first claw in the opening/closing direction is greater than a length between the first arm and the second arm at a time when the first arm and the second arm are in the closed state.
